(19) United States
(12) Patent Application Publication
(10) Pub. No.: US 2003/0176904 A1
(43) Pub. Date: Sep. 18, 2003

(54) Cold strip wrist band

(76) Inventor: Ardella Patterson, Plano, TX (US)

Correspondence Address: Ardella Patterson, 1629 Wagon Wheel Drive, Plano, TX, 75023, US

(21) Appl. No.: 10/099,394

(22) Filed: Mar. 15, 2002

(51) Int. Cl. A61F 5/00; A61F 7/00

(52) U.S. Cl. 607/108; 607/112; 607/114

(57) ABSTRACT

Developed in response to need of effective, long lasting, cooling therapy for women of menopause and for body overheating. This self-adhering cotton fabric cold strip is to be worn as a wrist band, or a ankle band. The self-adhering cotton fabric cold strip has an envelope defining a sealed cold strip volume. A cooling gelatinous material is positioned in the cold strip volume. A bandage sheet is fixed to the envelope. The bandage sheet defines a bandage adhesive for temporary adhesion of the cold strip to the skin surface of a user. The self-adhesive envelope contains a cooling gelatinous material which reduces the body temperature. The reduction of the body temperature brings relief that is convenient, portable, and lasts for hours. The cold strip is to be worn for a period of hours and is then disposed and replaced with another cold strip. The self-adhering cotton fabric cold strip will be produced with different colors of cloth. This will help the user from drawing unwanted or embarrassing attention to a therapeutic wrist or ankle band. Developed in response to need of effective, long lasting, cooling therapy for women of menopause and for body overheating. This self-adhering cotton fabric cold strip is to be worn as a wrist band, or a ankle band. The self-adhering cotton fabric cold strip has an envelope defining a sealed cold strip volume. A cooling gelatinous material is positioned in the cold strip volume. A bandage sheet is fixed to the envelope. The bandage sheet defines a bandage adhesive for temporary adhesion of the cold strip to the skin surface of a user. The self-adhesive envelope contains a cooling gelatinous material which reduces the body temperature. The reduction of the body temperature brings relief that is convenient, portable, and lasts for hours. The cold strip is to be worn for a period of hours and is then disposed and replaced with another cold strip. The self-adhering cotton fabric cold strip will be produced with different colors of cloth. This will help the user from drawing unwanted or embarrassing attention to a therapeutic wrist or ankle band.

### FEDERALLY SPONSORED RESEARCH

Not Applicable

### SEQUENCE LISTING OR PROGRAM

Not Applicable

### BACKGROUND

1. Field of Invention

This invention relates to the filed of medical devices. More particularly, this invention relates to the field of cold strips for application to the skin for reducing body temperature.

2. Description of Prior Art

It is well-known to apply cold packs to the skin surface of a user that has a fever. The cold pack can reduce fever. Conventionally, the cold pack, cooled by ice or activated chemicals, is applied to the forehead.

The cold pack is usually held in place by the patient receiving treatment. The requirement to hold the cold pack in position restricts the mobility of the patient and can in some circumstances be uncomfortable. Stretch wraps, tapes or other support materials have been improvised to support cold packs in location on the patient. Typically, these forms of improvised support materials are only appropriate for the limbs or head where the support material can encircle the patient to maintain the cold pack in position. Use of the cold pack in other portions of the body typically again requires the cold pack to be hand held into position.

Prior designs show a number of new cooling devices which include, for example, a plastic bag filled with a freezable liquid or gel. Such a cold pack is used in various therapeutic applications, as well as situations wherein overheating of an individual occurs during exercise or on hot and/or humid days. Some relevant art, however, employs devices which, can only be worn on the head or on the back of the neck, not allowing the wearing of the device in public. For a woman who has hot flashes throughout the day this is little help to her. Another disadvantage of the prior devices is that they are warmed in a short time, to a point such that their effective cooling becomes minimal to the wearer, they must then be taken off and disposed of.

Objects and Advantages

An object of the present invention is to cool an individual using a cold strip in a comfortable, portable, discreet fashion.

Yet another object is to provide a cold strip device which allows for substantially continuous cooling.

The invention offers relief for hot flash symptoms of menopause and body overheating by providing a wrist cooler, ankle cooler, chest cooler. The cooler preferably includes a cooling gelatinous material positioned in a cold strip volume. A woman experiencing symptoms such as menopausal hot flashes carries the strips in a pouch in her purse. Every four hours she places a strip around her wrist, or around her ankle, or under her clothes across her chest. This strip keeps her body temperature down, when a hot flash would occur she does not feel the effects or feels them very slightly, giving her much comfort and relief. The invention is easily transportable and provide for immediate relief.

I tested this process on myself, as a woman suffering through menopause and found it very effective and a wonderful relief to my hot flashes. It allowed me to rest at night without night sweats and to function at normal levels during the day without having to keep the temperature in my home so low it caused much discomfort to my family.

Hot flashes and similar symptoms can be experienced by women of all ages. Since women are living longer lives, symptoms such as hot flashes not only are more commonly encountered but also are being openly discussed and relief more actively pursued.

### SUMMARY

In accordance with the present invention a cotton fabric strip has a cooling gelatinous material positioned between the cotton fabric strip and the self adhesive with adhesive tab attaching it to the wrist or ankle.

### DRAWINGS

Drawing Figures

FIG. 1 shows a view of the invention as a self-adhering cold strip wrist band with the envelope of gelatinous material and the additional adhesive tab;

FIG. 2 shows a view of the invention as a self-adhering cold strip ankle band with the envelope of gelatinous material and the additional adhesive tab;

FIG. 3 shows the under-side view of the self-adhering cold strip wrist band/ankle band with the envelope of gelatinous material and a decorative pattern to the wrist band/ankle fabric and the additional adhesive tab;

FIG. 4 shows the upper-side view of the self-adhering cold strip wrist band/ankle band with a decorative pattern to the fabric;

FIG. 5 shows a cross-sectional view of the self-adhering cold strip wrist band/ankle band with the envelope of gelatinous material;

### REFERENCE NUMERALS IN DRAWINGS

| | | | |
|---|---|---|---|
| 8 | wrist of human hand | 16 | adhesive material |
| 10 | cotton fabric wrist band | 18 | gelatinous material |
| 12 | adhesive tab | 20 | ankle of human leg |
| 14 | ankle band | | |

### DETAILED DESCRIPTION

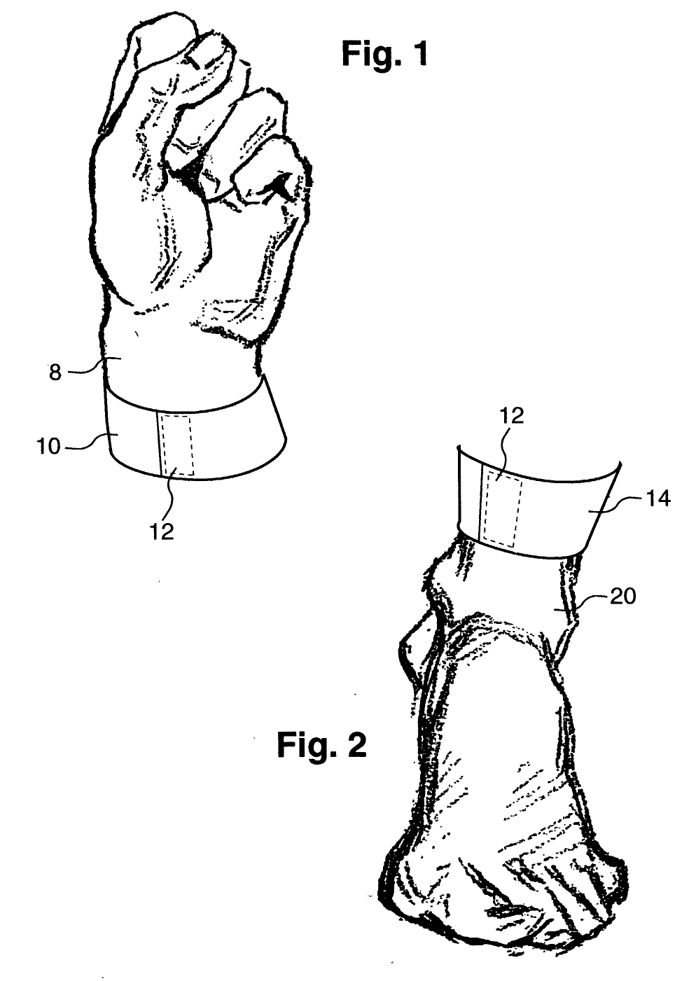

Description—FIG. 1

FIG. 1 shows a fabric self-adhesive wrist band (10) with cooling gelatinous material lying against all sides of the wrist (8) having an additional adhesive tab (12) positioned for lying against the wrapped upper side of the wrist band (10) the adhesive tab (12) closes over the wrist band (10) and adheres to the upper-side of the wrist band (10).

Description—FIG. 2

FIG. 2 shows an alternate form of the wrist band (10) as an ankle band (14). It shows a fabric self-adhesive ankle band (14) with cooling gelatinous material lying against all sides of the ankle (20) having an additional adhesive tab (12) positioned for lying against the wrapped upper-side of the ankle band (14). The adhesive tab (12) closes over the ankle band (14) and adheres to the upper-side of the ankle band (14).

The cooling gelatinous material that is adhered to the wrist band (10) fabric is held against the wrist by the self-adhesive material. Before the symptoms of discomfort occur, place the wrist band (10) or the ankle band (14) on the user. Placing the wrist band (10) or the ankle band (14) on the user before symptoms of discomfort proactively prevents the user's body temperature from rising to an uncomfortable level. After a number of hours of comfort, unwrap the wrist band (10) from the wrist (8) or unwrap the ankle band (14) from the ankle (20) and discard. Replace and wrap around the wrist (8) or the ankle (20) of the user a fresh, new wrist band (10) or ankle band(14).

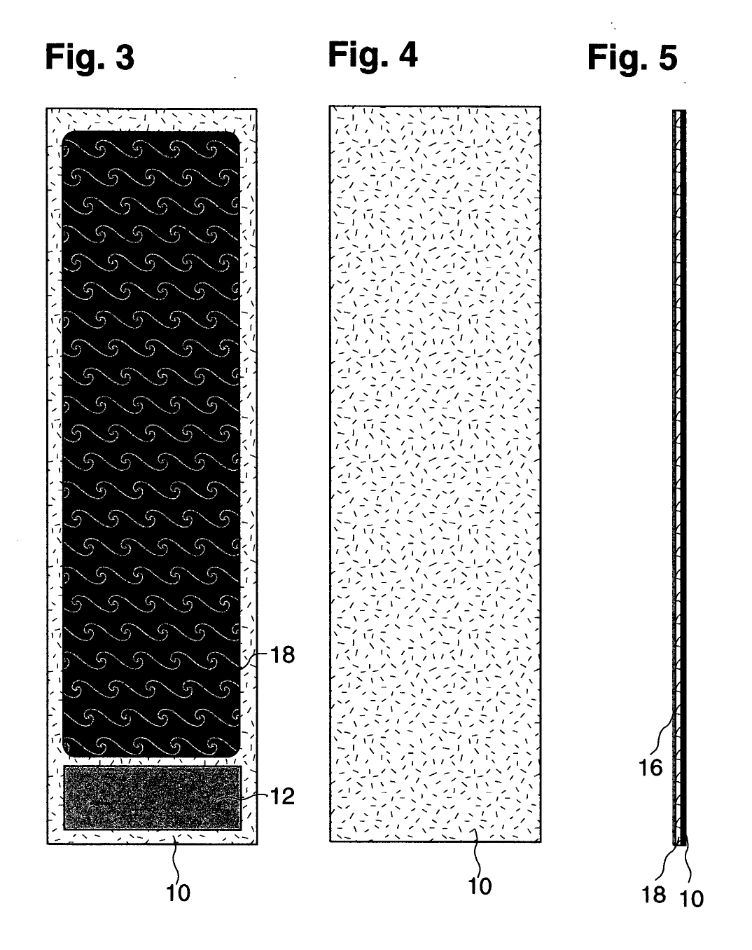

Description—FIGS. 3 and 4

The wrist band (10)/ankle band (14) shown in FIGS. 3 and 4 is a thin fabric wrist band (10) with a layer of self-adhesive cooling gelatinous material (18) spread across the wrist band (10) fabric and an additional adhesive tab (12) positioned at one end of the underside of the wrist band (10)

The wrist band (10)/ankle band (14) shown in FIGS. 3 and 4 may be made of an decorative material and may have a decorative design similar to designs found on fashionable clothing.

Description—FIG. 5

The cross-section of the wrist band (10)/ankle band (14) shown in FIG. 5 is flat, showing the cooling gelatinous material (18) sandwiched between the wrist band (10)/ankle band (14) and the self adhesive material (16).

### CONCLUSION

The invention is intended primarily for the non-pharmaceutical relief of hot flash symptoms due to menopause and body overheating, and may be easily and conveniently carried and used and replaced or discard after use. The preferred material of the wrist band (10) or ankle band (14) is a fabric material which is slightly stretchable to conform to the wrist (8) or ankle (20). The tab (12) and strip (16) are adhesive.

While preferred embodiments of the present invention have been illustrated and described in detail, it should be readily appreciated that many modifications and changes thereto are within the ability of those of ordinary skill in the art. Therefore, the claims are intended to cover any and all of such modifications which fall within the true spirit and scope of the invention.

I claim:

1. A self-adhering cotton fabric cold strip comprising:
a cooling gelatinous material in the strip volume; a flexible bandage adhesive sheet engaging the material and having additional securement adhesive means at one end of the perimeter edge; also adhesive means for mounting the cotton strip to the gelatinous material and for temporary constant adherence of the cold strip to a skin surface, reducing the body temperature of the user and causing cooling therapy.

2. The self-adhering cold strip of claim 1 wherein the cooling gelatinous material has a volume for the cooling gelatinous material reaching ambient temperature prior to over cooling of a skin surface.

3. The system of claim 1, wherein the cold strip has a self adhesive lower surface and an additional adhesive fastener at one end to attach to the opposite end so that the cold strip can be wrapped and self adhered around a user's wrist or ankle and fastened on itself.

4. The self-adhering cotton fabric cold strip of claim 3 wherein the bandage sheet has an upper surface and a lower surface, the adhesive is on the lower surface. The cooling gelatinous material is enveloped between the upper surface bandage sheet and the adhesive said lower surface.

5. This self-adhering cotton fabric cold strip of claim 3 is to be worn as a wrist band, or an ankle band.

6. This self-adhering cotton fabric cold strip of claim 1 is to be worn for a number of hours and then disposed of and replaced with another cold strip.

7. A method of claim 1 for treating a hot flash or sustaining a lower body temperature before a hot flash occurs, in order to reduce the effect of the hot flash to a warm flash or no flash at all.

8. The method of claim 3, further comprising wrapping the self adhesive strip around the user's wrist and attaching it to itself, so that the cold strip administers a cooling affect completely around the user's wrist or ankle.

9. A method of claim 1 for treating or reducing the body temperature of a Multiple Sclerosis patient, giving the patient energy. Active cooling improves fatigue and postural stability in MS patients.

10. A method of claim 1 for reducing body overheating due to exercise, working or playing outdoors.

11. The self-adhering cotton fabric cold strip of claim 1 will be produced with different colors of cloth. Natural colors, that will blend in with skin tones and designer colors such as blue, black, green, red, orange, and patterns to blend in with clothing. This will help the user from drawing unwanted or embarrassing attention to a therapeutic wrist band or ankle band.
